(19) United States
(12) Patent Application Publication
(10) Pub. No.: US 2004/0241114 A1
(43) Pub. Date: Dec. 2, 2004

(54) Hair Care and Nail Care Compositions Based on Ion-Pair Delivery System for Gender and Ethnic Selective Applications

(76) Inventor: SHYAM K. GUPTA, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/250,045

(22) Filed: May 30, 2003

(51) Int. Cl. A61K 7/04; A61K 7/06; A61K 7/11

(52) U.S. Cl. 424/61; 424/70.14; 424/74

(57) ABSTRACT

This invention relates to a novel ion-pair delivery system useful for gender and ethnic background selective hair care and nail care applications in which an electron donor composition and an electron acceptor composition, or a proton donor composition and a proton acceptor composition, or an anionic and a cationic composition, are combined synergistically. The bioavailability, deposition, functional performance, and consumer aesthetics of the compositions thus combined in such ion-pairs are enhanced synergistically. Hair care compositions, such as shampoo, conditioner, hair lotion, hair oil, hair gel, hair sheen, hair rinse, hair balm, hair wax, hair spray, and such, and nail care compositions, such as nail enamel, nail creams, nail serums, nail lacquers, nail spray, and nail polish, and such, can thus be obtained with synergistically enhanced performance.

### BACKGROUND OF INVENTION

In modern cosmetic science much attention has been bestowed upon skin care compositions relative to the alleviation of skin problems resulting from natural aging process, attack from UV and free radicals, and diseases such as psoriasis, eczema, acne and rosacea. The solution to the problems caused by similar factors to keratinous surfaces, such as human hair and nails, has not received as much attention in the prior art. For example, human hair is also susceptible to damage both from natural factors such as aging, UV, free radicals, and atmospheric pollutants; and from man-caused factors such as chemical bleaching agents, hair perming, and hair colorants, and such. These factors can cause hair thinning, hair loss, hair brittleness, loss of hair elasticity, graying, split-ends, loss of body and volume, and dull appearance, as further discussed in U.S. Pat. Nos. 6,557,562 (Rathnam) and 6,540,791 (Dias). Human nails become dull, brittle, chapped, and start to develop cracks under similar conditions of natural aging, attack from UV and free-radicals, and the effects of harsh cleansers, detergents, bleaches, and abrasives encountered in daily life.

As is well known in the current art, the hair morphology, chemistry, and grooming habits are different for male and female human gender and ethnicity. For example, relative to the hair's density, speed of growth or the implantation of the hair follicle in the scalp, it is well known that among three commonly recognized ethnic types, African, Asian, or Caucasian, each of the ethnic types mentioned has its own identity. Asian hair holds the speed record for growth, with 1.3 cm a month. On the other hand, it has lower density than any of the other ethnic groups. The way its follicle is implanted causes the hair to grow straight, perpendicular to the scalp. African hair is the slowest growing of all, at less than 0.9 cm a month. On the other hand it is slightly denser than Asian hair and grows almost parallel to the scalp, twisting around itself as it grows. As far as rate of growth is concerned, Caucasian hair comes between the other two at 1.2 cm a month, but it has the highest density of all. Also, it grows at an oblique angle to the scalp and is slightly curved.

Hair and nails contain keratin, a fibrous protein. Keratin is also the predominant protein in wool, horn, claws, and scales. Keratin contains all of the common amino acids, and differs from other structural proteins, such as skin proteins, chiefly by its higher cystine content. Hair thus contains a much larger proportion of sulfur amino acids, cysteine and cystine, compared to skin. Cystine is formed by the oxidative dimerization of cysteine. Cysteine is also a very good antioxidant due to its reducing nature (similar to ascorbic acid, which is also a very good antioxidant with chemical reducing properties). The degree of cross-linking of cysteine in hair determines hair's strength and curliness. The greater the cross-linking, usually higher the curling effect. In general, female human hair can contain less cross-linking of cystine than male human hair. Also, the Caucasian-type female human hair is less cross-linked than Negro-type female human hair. The compositions for hair care that are based on above stated scientific criteria, and consumer perception requirements of male and female human gender, are essentially non-existent in the prior art. To further illustrate, the damage from free radicals alone can cause cross-linking of cysteine moiety of hair protein forming a greater proportion of cystine, which can result in the loss of hair elasticity, curling of hair, and loss of silky appearance and flow. Also, both UV and free radicals can attack melanin in hair, resulting in hair graying, loss of hardness, strength, breaking strength, and tear strength or bundle tensile strength. These same factors can also attack hair dyes (deposited on hair during hair coloring process) and cause degradation of hair dyes resulting in hair discoloration and loss of shine. This is discussed in detail in U.S. Pat. No. 6,482,808 (Springob et al.): Various hair coloring methods themselves can also cause damage to keratin, as further discussed in U.S. Pat. Nos. 6,562,080 (Lim et al.), 6,554,870 (Cotteret et al.), and 6,479,042 (Nguyen et al). These problems have led to the development of certain keratin strengthening agents, as disclosed in U.S. Pat. No. 6,379,659 (Ishida et al.), and Springob et al.

It is thus of paramount importance that new ingredients and compositions selective for hair and nail care needs of human male and female gender and their ethnic backgrounds be developed. In combination with appropriate delivery systems, such ingredients can provide gender and ethnicity targeted hair and nail care compositions.

It is well recognized in the scientific community that delivery systems are highly useful in cosmetics and pharmaceutical disciplines. In a recent article written by present inventor (Cosmetic Delivery Systems, Household & Personal Products Industry, commonly known as HAPPI magazine, January 2003 issue, page 79) the definition and benefits of a number of prior art delivery systems have been discussed. A delivery system is thus a combination of both art and science that can improve the performance and consumer appeal of a consumer product or composition.

Surprisingly, I have now found that the combination of one hair care or nail care composition with another hair care composition or nail care composition by an ion-pair mechanism, as shown in Equation 1, provides a solution to this problem. The ion-pair combinations thus formed are more bioavailable, have selective but synergistic benefits, and are economical to produce from commonly available ingredients.

Electron Donor Composition+Electron Acceptor Composition â [symbol] Ion-Pair Composition (Equation 1)

For example, quaternary ammonium compounds are commonly used in modern hair care compositions for various benefits that include conditioning, shine, split-end treatments, hair repair, and such. These ammonium compounds, which are cationic in nature, also contain an anionic counter-ion as an ion-pair. For example, Crodasorb UV-HPP (Polyquaternium-59) is a -polymeric quaternary ammonium composition in which chloride and methosulfate are attached as anionic counter-ions. It is well appreciated by those who are versed in this art that only the cationic part of such quaternary ammonium compositions provides hair care benefits such as preventing damage by UV, and protection of tensile strength, hydrophobicity, combing properties, and natural color. In another example, Incroquat UV-283 (Cinnamidopropyltrimonium chloride), a UV-absorbing quaternary ammonium compound, provides protection to gray as well as tinted hair from damage by UV and free radicals. The hair strength and its mechanical behavior are also protected from damage by the same natural factors. In this example, the cationic Cinnamidopropyltrimonium moiety of this composition provides such benefits, and the anionic (chloride) part does not provide any hair beneficial effects. In these examples, if the anionic portions of these compositions were replaced by another anionic composition that has hair or nail beneficial properties, then such compositions could be more beneficial to hair and nails and that may also permit the custom designing of compositions selective for the needs of male or female human gender.

From the above discussion it should also be clear that sulfur amino acids, and compositions that contain a sulfhydryl (—SH), group are very important for human hair. The deposition of cysteine on hair can provide benefits such as antioxidant protection, more bioavailable sulfur for the synthesis of cystine, and such. Cysteine and its simpler derivatives, such as N-acetyl-cysteine, are water-soluble. The deposition of such water-soluble compositions on hair in any significant amount from a rinse-off consumer product, such as a shampoo or a body wash, has been difficult in the prior art. The combination of a cationic quaternary ammonium composition; for example, Cinnamidopropyltrimonium chloride (which is disclosed in U.S. Pat. Nos. 5,633,403 and 5,601,811) with an anionic cysteine derivative (Equation 2), surprisingly, solves this problem, which is further discussed in Example 5 of the present invention.

Cinnamidopropyltrimonium chloride+Sodium N-Acetyl-Cysteinate â [symbol] Cinnamidopropyltrimonium N-Acetyl-Cysteinate+Sodium Chloride (Equation 2)

In the above example, the preparation of Sodium N-Acetyl-Cysteinate is very simple from commonly available ingredients, as illustrated in Equation 3.

N-Acetyl-Cysteine+Sodium Hydroxide â [symbol] Sodium N-Acetyl-Cysteinate+Water (Equation 3)

The ion-pair compositions of the present invention can also be prepared by the combination of a proton-donating and a proton-accepting composition, as shown in Equation 4.

Proton Donor Composition+Proton Acceptor Composition â [symbol] on-Pair Composition (Equation 4)

To illustrate this point, glutathione, a sulfur peptide known to be hair beneficial antioxidant, and niacin, known to cause tingling effect on scalp, can be combined in an ion-pair mode to produce niacin glutathionate, a new ion-pair ingredient that has antioxidant and scalp tingling properties with better deposition on hair and scalp and thus better bioavailability, as shown in Equation 5.

Niacin+Glutathione â [symbol] Niacin Glutathionate (Equation 5)

Although not bound by any theory or hypothesis, it is also my belief that certain ion-pair delivery system of the present invention (such as those in Example 5) can provide additional benefits for the living portion of human hair, nails, and skin because the ion-pair undergoes ion-separation under the conditions of physiological pH (which is about 7.4), when such compositions are absorbed into hair shaft, nail, or skin, as shown in Equation 6.

Ion-Pair Composition â [symbol] Electron Donor Composition+Electron Acceptor Composition (Equation 6)

Such ion-separation of ion-pair composition releases the corresponding compositions in their active form and at the site of their action. For example, Niacin Glutathionate undergoes ion-separation under the conditions of physiological pH to release both Niacin and Glutathione in their original electronic states, as shown in Equation 7.

Niacin Glutathionate â [symbol] Niacin+Glutathione (Equation 7)

It is easily appreciated by those who are versed in the art that in cases where two ion-pairs are interacting with each other, new ion-pairs are formed on the basis of chemical reactivity principles by which a strong cation preferentially binds with a strong anion, and a weak cation preferentially binds with a weak anion. For example, in Equation 2, Cinnamidopropyltrimonium chloride contains Cinnamidopropyltrimonium cation, which is a weak cation, and chloride, which is a strong anion. In case of Sodium N-Acetyl-Cysteinate, Sodium is a strong cation, and N-Acetyl-cysteinate is a weak anion. By an ion-pair interchange reaction, which follows the ion-pair strength rule mentioned above, stronger sodium cation reacts with stronger chloride anion, and weaker Cinnamidopropyltrimonium cation reacts with weaker N-Acetyl-cysteinate anion, to form the ion-pair composition shown in Equation 2.

Regardless of the actual mechanism of the formation or the benefits caused by the ion-pair compositions prepared according to present invention, the gender selective benefits of such ion-pair compositions to male and female human hair and nails are unprecedented in the prior art.

### SUMMARY OF INVENTION

This invention relates to a novel ion-pair delivery system useful for gender and ethnic selective hair and nail care applications in which an electron donor composition and an electron acceptor composition, or a proton donor composition and a proton acceptor composition, or an anionic and a cationic composition, are combined synergistically. The bioavailability, deposition, functional performance, and consumer aesthetics of the compositions thus combined in such ion-pairs are enhanced synergistically. Hair care compositions, such as shampoo, conditioner, hair lotion, hair oil, hair gel, hair sheen, hair rinse, hair balm, hair wax, hair spray, and such, and nail care compositions, such as nail enamel, nail creams, nail serums, nail lacquers, nail spray, and nail polish, and such, can thus be obtained with much improved synergistic performance.

### DETAILED DESCRIPTION

This invention describes the combination of a cosmetic or pharmaceutical composition by using another cosmetic or pharmaceutical composition by an ion-pair mechanism in which one composition is an electron-donor or proton-acceptor, and the other composition is an electron-acceptor or proton-donor to form the ion-pair combinations (i.e. anionic-cationic pairs) that are more bioavailable, have synergistic hair care benefits, and are economical to produce from commonly available ingredients.

The ion-pair compositions thus formed are not just physical mixtures of the two compositions that are combined in an ion-pair mode to form such ion-pair compositions. Such ion-pair compositions are discreet chemical entities. For example, the combination of niacinamide with N-Acetyl-Cysteine results in the formation of ion-pair complex, Niacinamide N-Acetyl Cysteinate. In this example, niacinamide, which is a proton-acceptor composition, has the following properties: crystalline white powder, melting point 130Â° C., pH of 1% water solution=6.3, solubility in water=50%, stability of water solution=good. N-Acetyl-Cysteine, which is a proton-donor composition, has the following properties: crystalline white powder, melting point 110Â° C., pH of 1% water solution=3.5, solubility in water=>20%, stability of water solution=poor. Niacinamide N-Acetyl-Cysteinate, the ion-pair that is produced from the combination of the above two compositions is a new ingredient properties of which are very different from that of the above two compositions, which shall be described elsewhere.

The hair of male human has certain chemical, biochemical, personal grooming, and social acceptance features that are different from that of female human hair. The ion-pair compositions of the present invention are useful for gender selective hair and nail beneficial compositions.

It is clear from the prior art disclosures that sulfur amino acids, sulfur peptides, and sulfur proteins are essential part of human hair and nails. The strength, elasticity, natural coloration, volume, shine, and fragility are all dependent on, to a great extent, on the oxidation/reduction states of sulfur amino acids in hair and nails. While sulfur amino acids, such as cysteine and N-acetyl-cysteine, have been used in various skin care, and even in hair dye compositions (principally as reducing agents for hair dyes), these ingredients have not been disclosed in prior art for other hair and nail beneficial applications, particularly as ion-pair compositions in combination with other hair and nail beneficial ingredients To further illustrate the above point, U.S. Patent Application 20,030,095,933 (Klysz et al.) discloses N-acetyl-cysteine in antiaging skin care compositions. U.S. Patent Application 20030087819 (Bielicki et al.) discloses certain cysteine peptides in antioxidant applications. U.S. Patent Application 20030003148 (Guo et al) disclose that N-acetyl cysteine can be used as a scavenger to prevent cell death due to free radicals, and to prevent nitrate intolerance following chronic treatment with nitroglycerine and sustained release nitrate formulations. N-acetyl cysteine also has value as a nutritional supplement. However, it also has an extremely bad taste that per se creates a great problem in administering it. This would indicate that topical delivery systems with this ingredient might be more suitable for such applications. U.S. Patent Application 20020115723 (Iwasaki et al.) disclose a method for preventing, retarding, alleviating or treating a skin change due to aging or an undesirable aesthetic skin change, both caused or promoted by oxidative stress, utilizing cysteine derivatives. U.S. Patent Application 20020090670 (Malee et al.) discloses the application of certain cysteine peptides to control topical oxidative damages. Both glutathione, a sulfur peptide present ubiquitously in human body, and cysteine derivatives have been disclosed in U.S. Patent Application 20030069311 (Herzenberg et al.) in certain drug toxicity reducing compositions. U.S. Pat. No. 6,531,608 (Pearson et al.) describes cysteine, and several other thiols, for antiaging and other applications. Cysteine is also a biochemical precursor to glutathione (U.S. Patent Application 20030054048).

Sulfur compounds, especially those with free sulfhydryl (-SH) group, are known to bind with skin and hair and impart certain beneficial properties. Methylthioadenosine has thus been used for enhancing pigmentation in skin and hair (U.S. Pat. No. 6,551,581). N-Acyl-amino acid esters have found application in skin care compositions (U.S. Pat. No. 6,528,068).

One of the most interesting applications of sulfhydryl chemistry is the oxidative disulfide formation from cysteine derivatives (U.S. Patent Application 20,020,082,384, Verdini et al.). This oxidation can be carried out even by air, and the disulfide derivatives thus formed undergo protein-folding process to produce proteins that can mimic natural proteins. This would suggest that if cysteine and its derivatives were deposited on hair and nails from rinse-off products, then they may undergo similar disulfide formation with cysteine or other sulfhydryl compositions already present on hair or nails. The formation of such cross-linked disulfide bonds would provide additional strength to hair and nails, and also increase their tensile strength. The teachings of the present invention provide such practical solutions to solve the problems of hair and nails. Moreover, the delivery of such sulfhydryl compositions, even those that are water-soluble, can be achieved even from rinse-off hair and nail care compositions, as disclosed in the present invention.

Furthermore, it has been reported that certain derivatives of cysteine, such as N-acetyl-cysteine, are unstable in certain compositions (U.S. patent application Ser. No. 20020037855, Stanislaus); such derivatives are stable and easy to formulate by the process of the present invention, as further illustrated in the Examples section of the present invention.

Most hair and nail beneficial sulfur compounds mentioned above, such as cysteine, cystine, N-acetyl-cysteine, biotin, glutathione, and such, are water soluble. These ingredients are not easily deposited in any appreciable or beneficial amounts from rinse-off compositions such as shampoo or conditioner. The combination of such water soluble ingredients with another hair or nail beneficial composition that is known to be deposited on hair and nails (by the attractive forces of cationic and anionic electrical charges) in significant amounts from aforementioned rinse-off products has provided a new method for the deposition of such water soluble ingredients in the present invention. This is both surprising and unexpected because such combination of cationic and anionic compositions usually renders both of such combining compositions ineffective due to the self-neutralization of their positive and negative charges. For example, the combination of stearalkonium chloride, a very commonly used hair conditioning agent that is also a quaternary ammonium composition (i.e. cationic in nature), upon combination in an ion-pair mode with an alkali metal derivative of a carboxylic acid, such as sodium cocoate (which is anionic in nature) forms the ion-pair derivative, stearalkonium cocoate, which does not have any hair conditioning benefits at all due to internal neutralization of cationic and anionic charges. Surprisingly, a similar combination of stearalkonium chloride with sodium N-acetyl-cysteinate (also an alkali metal derivative of a carboxylic acid) results in the formation of stearalkonium N-acetyl-cysteinate, which does have both the hair conditioning benefit of stearalkonium cation and antioxidant benefit of N-acetyl-cysteinate anion.

The compositions of the present invention, unexpectedly, do not suffer from such disadvantages, and provide a dual benefit, first from the ingredient that is known to be deposited on hair or nails (i.e. cationic moiety) and, second from the water soluble ingredient (anionic moiety) that was attached to the first composition by an ion-pair delivery system. Although certain quaternary ammonium compounds have been disclosed in the prior art, for example, as penetration enhancers in certain transdermal delivery systems (U.S. Patent Application 20030091620, Fikstad et al.), as hair substantive compositions (U.S. Patent Application 20030064083, Koshti et al., and 20030036665, Walele et al.), and as pharmaceutical delivery systems (U.S. Patent Application 20030065015, Bacaner et al.), the aforementioned benefits are mostly from the surfactant action of such quaternary ammonium compounds, and not from their combination in an ion-pair mode with another composition.

The ion-pair compositions of the present invention can be made by the reaction of an electron donating (ED) and electron accepting (EA) compositions, or by the reaction of a proton donating (PD) and a proton accepting (PA) composition, wherein the two such reacting electrically opposite compositions are present in an equimolar to less than equimolar ratios. For example ED can be equimolar, or less than equimolar amounts than EA. EA can be in equimolar, or less than equimolar amounts than ED. Similar molecular equivalence rations also apply to PD and PA in such ion-pair forming reactions.

### EXAMPLES

The following examples are presented to illustrate presently preferred practice thereof. As illustrations they are not intended to limit the scope of the invention. All quantities are in weight %. The examples illustrate specific compositions for men' hair applications. This is because the prior art has ignored compositions specific for men's hair applications. However, this does not limit the use of the compositions of the present invention in women's hair applications.

### Example 1

### Preparation of Sodium N-Acetyl-Cysteinate

(1) N-Acetyl-Cysteine 15.1 (2) Deionized Water 76.9 (3) Sodium Hydroxide (50% solution) 8.0 Procedure. Mix (1) and (2) to a clear solution. Add (3) and mix. A clear, 17.3% solution of Sodium N-Acetyl-Cysteinate is obtained. On evaporation of water under vacuum, a white solid crystalline composition of Sodium N-Acetyl-Cysteinate (100% molecular activity level) is obtained.

### Example 2

### Preparation of Cinnamidopropyltrimonium N-Acetyl-Cysteinate

(1) Sodium N-Acetyl-Cysteinate 17.3 (100% molecular activity level) (2) Cinnamidopropyltrimonium chloride (Molecular Weight 283, molecular activity level 70%) 40.4 (3) Deionized water 42.3 Procedure. Mix (1) and (2) to a clear solution. Add (3) and mix. A light amber solution is obtained, which contains 39.7% of Cinnamidopropyltrimonium N-Acetyl-Cysteinate.

### Example 3

### The In-Situ Preparation of a 32.8% High Potency Niacinamide Lipoate hair and Nail Serum from Niacinamide and Lipoic Acid

(1) Lipoic Acid 20.6 (2) Niacinamide 12.2 (3) Deionized Water 33.2 (4) Propylene Glycol 34.0 (5) Niacinamide Lipoate 32.8. Procedure:

All ingredients from (1) to (4) were mixed and heated at 40 to 50 C. for 30 minutes. The product was cooled. A thin solution of composition that was a mixture of (3) to (5) was obtained.

### Example 4

### The In-Situ Preparation of a 42.9% High Potency Niacinamide Glutathionate Antioxidant Hair Spray Composition

(1) Glutathione 30.7 (2) Niacinamide 12.2 (3) Deionized Water 50.0 (4) Methylpropanediol 7.1 (5) Niacinamide Glutathionate 42.9. Procedure: All ingredients from (1) to (4) were mixed. A solution of Niacinamide Glutathionate of the composition that was a mixture of (3) to (5) was obtained.

### Example 5

### Hair Deposition Evaluation of Compositions from Example 1 and Example 2

Human hair (about 10 cm long) was obtained from 10 individuals (five male and five female) from the barbershops. It was washed with water and air dried. Portions of these hair samples were soaked for one hour in (1) distilled water, (2) in a solution obtained from Example 1, and (3) in a solution obtained from Example 2.

These hair swatches were then removed and rinsed with distilled water. These hair swatches were then immersed in a 1% solution of sodium hydroxide. The hair swatches were then removed, and the sodium hydroxide solution was first heated at 70 to 80 C. for 1 hour to deacetylate N-acetyl-cysteine, then neutralized with hydrochloric acid to pH 5.5 and reacted with ninhydrin reagent solution. The blue color was obtained in samples from (2) and (3) above. The spectroscopic measurement showed that only about 1% of N-acetyl-cysteine was deposited on hair from sample (2). The deposition of N-acetyl-cysteine from sample (3) was at least 10%. These data are from the averages obtained from the hair of 10 individuals noted above.

### Example 6

### Preparation of Polyquaternium-59 Glutathionate

(1) Polyquaternium-59 10.0 (65% active level commercial solution) (2) Deionized water 85.4 (3) Glutathione 3.0 (4) Sodium Hydroxide 1.6. Procedure. Mix (2) and (3) to a clear solution. Add (4) and mix. Add (1) and mix. A light brown solution of Polyquaternium-59 Glutathionate (about 8.0% “active” in water) is obtained.

### Example 7

### Men's Shampoo With Free-Radical Protection and Hair Strength Builder Compositions

(1) Sodium Lauryl Ether Sulfate 35.0 (2) Deionized Water 57.0 (3) Cinnamidopropyltrimonium N-Acetyl-Cysteinate 5.0 (4) Preservatives 0.5 (5) Laureth-3 2.5 Procedure. Mix (1) to (4) to a clear solution. Add (5) to thicken. A shampoo composition with viscosity of 9,000 cps and pH of 5.5 is obtained.

### Example 8

### Men's Hair Conditioner with a Sulfur-Source and Antioxidant Compositions

(1) Deionized Water 82.5 (2) Behentrimonium methosulfate 6.0 (3) Cetyl alcohol 2.0 (4) Stearalkonium chloride 2.0 (5) Polyquaternium-59 Glutathionate 5.0 (6) Methylsulfonylmethane (MSM) 2.0 (7) Preservative 0.5 Procedure. Mix (1) to (4) and heat to 60 to 70 C. Cool to 35 to 40 C. and add (5) to (7) and mix. A creamy, lotion like composition is obtained.

### Example 9

### The In-Situ Preparation of a 36.6% High Potency Niacinamide Biotinate Hair and Nail Serum from Niacinamide and Biotin

(1) Biotin 24.4 (2) Niacinamide 12.2 (3) Deionized Water 53.4 (4) Propylene Glycol 10.0 (5) Niacinamide Biotinate 36.6. Procedure: All ingredients from (1) to (4) were mixed and heated at 40 to 50 C. for 30 minutes. The product was cooled. A thin solution of composition that was a mixture of (3) to (5) was obtained.

### Example 10

### Nail Hardening, Shine, and Protectant Composition

(1) Deionized water 40.5 (2) Denatured alcohol 50.00 (3) Shellac 5.0 (4) Bamboo Extract 2.0 (5) Niacinamide Glutathionate 1.0 (6) Niacinamide biotinate 0.5 (7) Tolnaftate 1.0. Procedure. Mix (2), (3), and (7) to a clear solution. Mix separately (1), (4), (5), and (6) ingredients to a solution. Mix these two solutions. A light amber solution is obtained.

I claim:

1. The preparation of hair care and nail care ion-pair compositions in which;
(i) An electron accepting composition and an electron donating composition; or a proton accepting composition and a proton donating composition, are combined by an electron-proton ion-pair bonding (or, anion-cation bonding) mode, and
(ii) Both electron donating and electron accepting compositions; or proton accepting and proton donating compositions; or anion-cation pairs, have hair and nail beneficial attributes that are independent of each other, and
(iii) The electron donating and electron accepting compositions; or proton accepting and proton donating compositions; or anion-cation pairs, have synergistic hair and nail care benefits, when combined in ion-pair composition, and also when released from such ion-pair composition under the conditions of physiological pH, and
(iv) A cosmetically or pharmaceutically acceptable carrier base composition.

2. A composition according to claim 1, wherein proton donating composition in the ion-pair can be a sulfur-rich amino acid or sulfur-rich peptide derivative or composition.

3. A composition according to claim 1, wherein electron donating composition in the ion-pair can be an organic amine derivative or composition.

4. A composition according to claim 1, wherein a cationic composition in the ion-pair can be a quaternary ammonium derivative or composition.

5. A composition according to claim 1, wherein an anionic composition in the ion-pair can be sulfur-rich carboxylate, phosphate, or phosphonate derivative or composition.

6. The compositions according to claim 1, wherein the cosmetically or pharmaceutically acceptable delivery system can be traditional water and oil emulsions, suspensions, colloids, micro emulsions, clear solutions, suspensions of nanoparticles, emulsions of nanoparticles, or anhydrous compositions.

7. A composition according to claim 1, wherein cosmetically or pharmaceutically acceptable delivery system or carrier base can optionally include additional hair, nail, and skin beneficial ingredients selected from cleansers, surfactants (cationic, anionic, non-ionic, amphoteric, and zwitterionic), conditioning agents, keratin synthesis boosters, keratin protectors, vitamins, hormones, minerals, plant extracts, anti-inflammatory agents, antimicrobial agents, antifungal agents, keratolytic agents, drug active agents, concentrates of plant extracts, emollients, moisturizers, hair, nail, and skin protectants, humectants, silicones, skin soothing ingredients, analgesics, hair, nail, and skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof. The quantities of such ingredients can be safe and effective amounts as needed, and not limited to any specific limits.

8. A composition according to claim 2, wherein proton donating composition can be further selected from, but not limited to, Cysteine, N-Acetyl-Cysteine, N-Acyl-Cysteine, N-Aroyl-Cysteine, Cystine, N-Acetyl-Cystine, N-Acyl-Cystine, N-Aroyl-Cystine, Taurine, N-acetyl-taurine, N-acyl-taurine, N-aroyl-taurine, Glutathione, Glutathione dimer (oxidized glutathione), Glutathione Mono Esters, Glutathione dimer esters, Lipoic acid, biotin, and combinations thereof.

9. A composition according to claim 3, wherein electron donating composition can be further selected from, but not limited to, allantoin, arginine esters, lysine esters, cysteine esters, cystine esters, DNA, RNA, Adenosine, Guanosine, Cytosine, niacinamide, methyl niacin, benzyl niacin, lauryl niacin, pyridoxine, pyridoxal, pyridoxamine, yohimbine, vincamine, vinpocetin, glucosamine, chitosan, chondroitin, glutathione, carnosine, creatine, creatinine and their derivatives, MSM, DMSO, and combinations thereof.

10. A composition according to claim 4, wherein a cationic composition can be further selected from, but not limited to, Stearalkonium chloride, Cetrimonium chloride, behentrimonium methosulfate, Dicetyl dimonium chloride, Benzalkonium chloride, various Polyquaternium compositions (such as Polyquaternium-1, Polyquaternium-2, Polyquaternium-3, Polyquaternium-4, Polyquaternium-5, Polyquaternium-6, Polyquaternium-7, Polyquaternium-10, Polyquaaternium-11, Polyquaternium-16, Polyquaternium-44, Polyquaternium-46, Polyquaternium-59, etc.), various UV-absorbing quaternium compositions (such as Cinnamidopropyltrimonium chloride), various Quaternium compositions (such as Quaternium-82, Quaternium-7, Quaternium-10, Quaternium-79, and Quaternium-79 condensates, etc.), silicone quaternium compositions, and combinations thereof.

11. A composition according to claim 5, wherein an anionic composition in the ion-pair can be further selected from, but not limited to, alkali, alkaline earth, or ammonium derivatives of cysteine, N-Acetyl-Cysteine, N-Acyl-Cysteine, N-Aroyl-Cysteine, Cystine, N-Acetyl-Cystine, N-Acyl-Cystine, N-Aroyl-Cystine, Glutathione, Glutathione dimer (oxidized glutathione), Glutathione Mono Esters, Glutathione dimer esters, biotin, lipoic acid, and combinations thereof.

12. A composition according to claim 7, wherein hair and nail beneficial ingredients that can be further selected from a group consisting of, but not limited to, antioxidants, collagen and elastin synthesis boosters, various hydroxy acids (alpha hydroxy acids, beta hydroxy acids, and polyhydroxy acids), vitamins, hormones, hair, nail, and skin brightening agents, UVA/UVB sunscreens, antimicrobial agents, antifungal agents, keratolytic agents, blood microcirculation improvement agents (vasodilatory or vasoconstrictive), hair and skin protectant drug actives, and combinations thereof. The quantities of such compositions can be safe and effective amounts as needed, and not limited to any specific limits.

13. A composition according to claim 7, wherein the composition to promote or protect keratin in hair and nails can be further selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), Î±-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.

14. A composition according to claim 7, wherein hair, nail, or skin beneficial hydroxy acid can be selected from the group consisting of, but not limited to, salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, and hyaluronic acid (HYA). The quantities of such compositions can be safe and effective amounts as needed, and not limited to any specific limits.

15. A composition according to claim 7, wherein the hair, nail, or skin beneficial brightening agent can be further selected from, but not limited to, hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

16. A composition according to claim 7, wherein hair, nail, or skin beneficial antioxidant composition can be selected from, but not limited to, Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

17. A Composition According to claim 7, wherein the blood microcirculation improvement composition can be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-vinifera) leaves, apigenin, phytosan, luteolin, and combinations thereof. The quantities of such compositions can be safe and effective amounts as needed, and not limited to any specific limits.

18. A composition according to claim 7, wherein the vitamin composition can be selected from, but not limited to, Vitamin A, members of Vitamins B group, Vitamin C, Vitamin D, Vitamin E, Vitamin K, Carotenes, Biotin, Folic Acid, and combinations thereof. The quantities of such ingredients can be safe and effective amounts as needed, and not limited to any specific limits.

19. A composition according to claim 7, wherein the delivery system or carrier base can additionally contain human gender selective and ethnic background selective hair and nail beneficial compositions that are based on scientific or folkloric considerations.
